(19) United States
(12) Patent Application Publication
(10) Pub. No.: US 2007/0154445 A1
(43) Pub. Date: Jul. 5, 2007

(54) Compositions, methods and kits for preventing the decomposition of urine

(76) Inventor: Paul Albert Smith, Golden, CO (US)

Correspondence Address: MARTIN & HENSON, P.C., 9250 W 5TH AVENUE, SUITE 200, LAKEWOOD, CO, 80226, US

(21) Appl. No.: 11/327,239

(22) Filed: Jan. 5, 2006

(51) Int. Cl. A61L 9/01

(52) U.S. Cl. 424/76.1

(57) ABSTRACT

The present disclose relates to a method for preventing the decomposition of urine. The method includes adding a chemical to a liquid urine solution in a collection device to alter the pH of the resulting solution to a level that substantially prevents or significantly reduces the metabolism and reproduction of bacteria such as Escherichia coli, Proteus vulgaris, and Pseudomonas. The chemical composition may be an acid, such as a particulate, non-toxic, food grade acid that lowers pH to less than about 5.5, but more specifically to less than 4.0. The chemical composition may be an alkaline compound, such as potassium hydroxide, that raises pH to more than about 11.5. This method may be implemented so that urine is treated in the collection container for a patient concurrently with the urine collection. A kit implementing the method is disclosed.

### BACKGROUND OF THE DISCLOSURE

This invention broadly relates to the treatment of urine to reduce odor. Thus, this disclosure involves compositions, methods and kits that may be employed to reduce the unpleasant smell of urine that may otherwise arise after a period of time following discharge for an animal or human. One aspect of the embodiments is the use of a chemical means for preventing a liquid solution containing urine from decomposing while being stored in typical room temperature conditions.

One potential application is a bedside urine collection device. Such collection devices are used by urostomy patients. If untreated, the urine solution in such a collection device becomes a culture medium for bacteria that decompose urea into foul-smelling ammonia. Other compounds in the urine may also be decomposed to produce additional foul odors. Another potential application is the treatment of pet or other animal urine on floors, carpets, etc., to reduce the incidence of foul smell resulting therefrom.

It is known that the odors from urine can be reduced or eliminated by masking them, by keeping the odors in a sealed vessel, by preventing the urine from decomposing, or by a combination of these techniques. Urine is relatively odorless if the organic chemicals it contains are prevented from breaking down. It is known that bacteria and other microbes cause the decomposition process in urine that generate the foul odors we associate with urine.

Prior art inventions have applied the use of acids to reduce odors produced by animals that urinate on a carpet, in a litter box, or on the bottom of a cage. This prior art relates to absorbents and surface treatments. In these cases, the urine mixes with the acid and the resulting mixture dries to a solid when the water and other liquids are exposed to open air. These methods are relatively crude and not directly applicable to eliminating odors from liquid urine solutions in a vessel.

It is not known, however, to inhibit the growth of microorganisms and prevent the creation of foul-smelling urine byproducts by either lowering or elevating the pH of urine by adding a small quantity of a chemical to a liquid urine solution to generate a specific range of acidity or alkalinity. It is also not known to use non-neutral pH to kill the odor-causing microbes in a urostomy application.

Therefore, there is a need for a simple chemical composition that may be employed to prevent the generation of odors in urine that is stored at room temperatures, especially in urostomy applications. There is a further need for methods of reducing the incidence of odors generated by the decomposition process in urine. There is a particular need for such compositions and methods that may be adapted to bedside overnight urine collection devices used by urostomy patients. The ideal method, system, and/or chemical composition should be safe, effective, low cost, easy to use, and available, for example in a kit, through mail order or over the counter. Such product and method should be safe and non-toxic to humans and other animals yet should be fatal to bacteria.

### SUMMARY OF THE DISCLOSURE

It is an aspect of the invention to provide new and useful compositions, methods and kits for use in reducing odors resulting from decomposition of urine.

An aspect of the exemplary embodiments is to provide compositions and methods for altering the pH of a volume of urine to a level the reduces or substantially prevents the growth of microbes, such as Escherichia coli (E. coli), Proteus vulgaris, or Pseudomonas, that survive and/or multiply in urine at room temperature conditions.

Another aspect of the exemplary embodiments is to provide a kit and method to reduce decomposition odors that may be adapted to bedside overnight urine collection devices used by urostomy patients.

In addition, it is an aspect of the exemplary embodiments to provide methods and compositions that are safe, effective, low cost, easy to use, and available, for example in a kit, through mail order or over the counter.

According to the general embodiment disclosed herein, a method for reducing decomposition odors in a volume of liquid urine solution is disclosed wherein a volume of liquid urine solution is mixed with a selected quantity of a compound to form a treated volume such that any significant growth of bacteria selected from a group consisting of Escherichia coli, Proteus vulgaris and Pseudomonas in the treated volume is inhibited. The composition may be an acidic compound, such as a non-toxic, food grade acid in particulate form, wherein the selected quantity is such that the treated volume is reduced to a pH of less than about 5.5, and more specifically, 4.0. This acid may be, for example, selected from a group consisting of citric acid, succinic acid, tartaric acid and mixtures thereof. Alternatively, the compound may be an alkaline compound, such as potassium hydroxide, wherein the selected quantity is such that the treated volume has a pH of more than about 11.5. A fragrance may also be introduced into the liquid urine solution, if desired.

Furthermore, a method of controlling decomposition odors during the collection of urine from a human patient is also contemplated by this invention. As discussed in the exemplary embodiments, this method may include, in any order, the steps of: (1) connecting the patient to a urine collection system having a collection container whereby production of urine from the patient will be collected and stored in the collection container; and (2) placing in the collection container a selected quantity of a compound to form a treated volume such that any significant growth of bacteria selected from a group consisting of Escherichia coli, Proteus vulgaris and Pseudomonas in the treated volume is inhibited. This method then includes the step of allowing the production of urine from the patient to be collected and stored in the collection container whereby, as said urine is collected and stored, said compound mixes with said urine. In this method, the compound may be selected to be an acidic compound as described above wherein the selected quantity is such that a volume of urine of no more than 600 milliliters when mixed with the compound will have a pH of less than about 5.5, or more specifically a pH of less that about 4.0. Alternatively, the compound may be an alkaline compound as described above wherein the selected quantity is such that a volume of urine of no more than 600 milliliters when mixed with the compound will have a pH of more than about 11.5. In this method, a fragrance may also be introduced into the liquid urine solution.

According to one aspect of the exemplary embodiments, a kit is disclosed for use in home or in a medical care facility. Here, the kit includes a container having a lid and a quantity of a compound therein that is either an acidic compound or an alkaline compound. A scoop is provided that has a selected volume “V” such that the volume nV of said compound, where n is 1 or 2, when mixed with about 600 milliliters of human urine, will form a treated volume of urine that has a pH of less than about 5.5 or more than about 11.5. More particularly, the volume nV of said compound will form a treated volume of urine that has a pH of less than about 4.0 or more than about 12.0. These compounds are as described above, including optional fragrances.

In addition, the invention contemplates compositions adapted to be placed in a volume of collected urine. These compositions generally are in the form of an admixture of a non-toxic, food grade acid compound and a fragrance wherein said admixture is in a dry particulate form and is substantially soluable in an aqueous environment. The non-toxic, food grade acid is selected from a group consisting of citric acid, succinic acid, tartaric acid and mixtures thereof.

These and other aspects of the present invention will become more readily appreciated and understood from a consideration of the following detailed description of the exemplary embodiments of the present invention when taken together with the accompanying drawings, in which:

### BRIEF DESCRIPTION OF THE DRAWINGS

The present disclosure is described in conjunction with the appended figures:

FIG. 1 shows a linear plot of the relationship between bacterial count of a urine sample and pH;

FIG. 2 shows a logarithmic plot of the relationship between bacterial count of a urine sample and pH; and

FIG. 3 shows a urine decomposition prevention kit;

In the appended figures, similar components and/or features may have the same reference label. For items with the same reference label, the description is applicable to any one of the similar components.

### DETAILED DESCRIPTION OF EMBODIMENTS

The ensuing description provides preferred exemplary embodiment(s) only, and is not intended to limit the scope, applicability or configuration of the invention. Rather, the ensuing description of the preferred exemplary embodiment(s) will provide those skilled in the art with an enabling description for implementing a preferred exemplary embodiment(s) of the invention. Specific details are given in the following description to provide a thorough understanding of the embodiments. It should be understood, however, that various changes may be made in the function and arrangement of elements without departing from the spirit and scope of the invention as set forth in the appended claims and that the embodiments maybe practiced without these specific details.

According to the exemplary embodiments of the method, the present disclosure prevents or reduces the decomposition of urine and its associated foul odor by preventing the metabolism, growth, and/or reproduction of bacteria and other microbes. If microbes cannot survive in a solution or solid containing urine, the urine cannot be converted to ammonia, the primary odor-causing chemical that one associates with the smell of urine. By making the urine-containing substance inhospitable to bacteria, the urine does not decompose and odors are minimized.

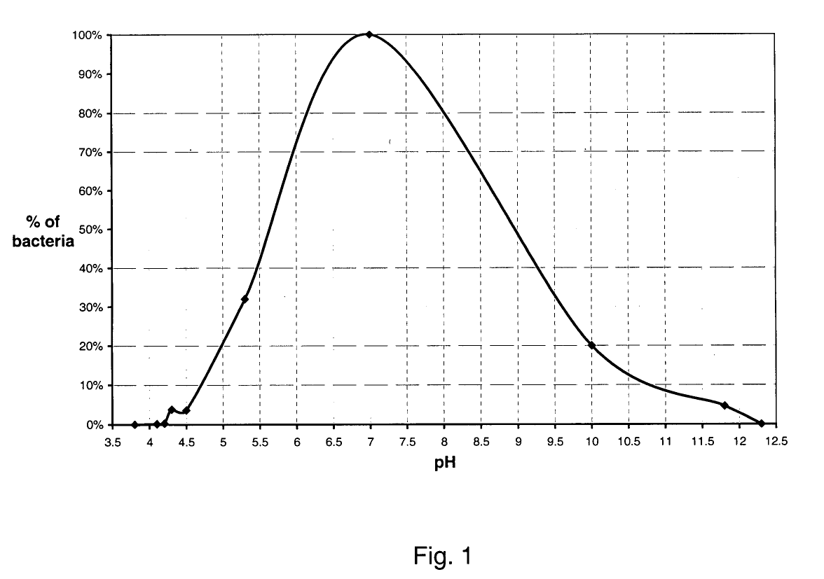

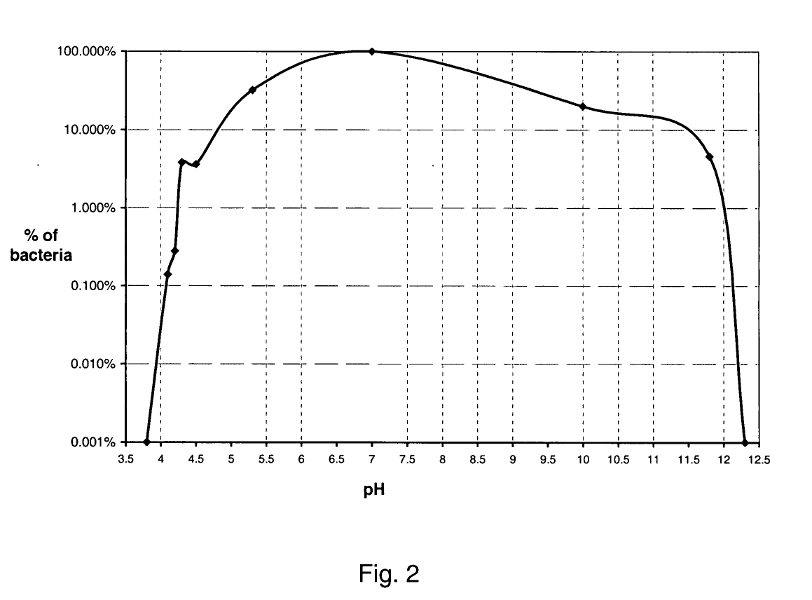

Referring to FIG. 1 and FIG. 2, the relationship between the pH of a urine solution and the survival and growth rates for common bacteria are shown on a linear graph and logarithmic plot respectively. This data was experimentally determined by inoculating human urine with live cultures of bacteria, namely, Escherichia coli (E. coli), Proteus vulgaris, and Pseudomonas. These three bacteria were chosen as being representative of the types of bacteria in our environment that thrive on breaking down urea.

After initial inoculation of a quantity of human urine with the bacteria, the inoculated urine solution was then divided into smaller samples. These smaller samples were then treated by adding an acid or a base composition thereby modifying the pH of the samples. The treated urine samples with various pH values were then allowed to grow bacteria at room temperature conditions for a growth interval of between seven hours and seven hours, twenty minutes. After the growth interval, each treated sample was then diluted with water, and a small quantity of each of the diluted liquids was placed on separate agar plates and allowed to harden.

The agar plates were then incubated for 24 hours during which time any bacteria in the dilution would grow into bacterial colonies that could be seen and counted. A bacterial count was then made for each plate. The resulting bacterial count for each sample was normalized in comparison with the bacterial count found in the untreated neutral urine having a pH of 7 to create the data shown on these two graphs.

With continued reference to FIG. 1 and FIG. 2, these experimental results show that maintaining a pH of less than 5.5 reduces the bacterial count to less than 50%, that maintaining a pH of less than 4.2 reduces bacterial count by approximately 100× and maintaining a pH of less than 4.0 reduces bacterial count by over 1000× in comparison to untreated urine. At the pH of about 4.0, this effectively means that all bacteria have been killed. By making the treated volume even more acidic by driving pH to less than 3.8, less than 3.6, less than 3.4, or less than 3.2, one can be extra certain that odor-causing bacteria cannot effectively survive.

Similar reductions in bacterial growth can be achieved by making the treated volume alkaline. By maintaining a pH of greater than 9, the bacterial count is reduced to less than 50%, by maintaining a pH of greater than 10, the bacterial count is reduced by a factor of more than 10×, by maintaining a pH of greater than 11.5, bacterial count is reduced to less than 8%, by maintaining a pH of greater than 12.0 reduces bacterial count by 100× and maintaining a pH of greater than 12.2 reduces bacterial count by over 1000× in comparison to an untreated urine solution. By driving pH even higher, to greater than 12.4, greater than 12.6, greater than 12.8, or greater than 13.0 one can be extra certain that odor-causing bacteria cannot survive in the urine solution.

It is well known that-the typical overnight output of urine from a human is about 200 milliliters, based on a minimum human urine output of 30 milliliters per hour, with a typical maximum overnight output being about 600 milliliters. Accordingly, various compositions were tested to determine their effect on the pH when added to 600 milliliters of urine. These compositions were selected since they are each non-toxic and can be made from food-grade chemicals. Moreover, each of these compositions are made from food-grade chemicals in dry particulate form that are safe to ship through the US Mail, through ground-based carriers such as the United Parcel Service, or through air carriers such as Federal Express. Table 1 listed selected examples of chemical compositions that drive pH low enough to be effective in preventing the decomposition of 600 milliliters of liquid urine. Other food-grade chemicals in dry or other form can be used in ways capable of being understood by anyone skilled in the art.

TABLE 1

| Chemical Composition and Amount | |
|---|---|
| Added to 600 Milliliters of Urine | Resulting pH |
| Pure citric acid in granular form (5 grams) | 3.7 |
| Pure succinic acid in granular form (5 | 4.2 |
| grams) | |
| Pure tartaric acid in granular form (5 | 3.3 |
| grams) | |
| Mixture of citric acid (3.5 grams) and | 3.5 |
| tartaric acid (1.5 grams) | |
| Mixture of citric acid (2.5 grams) and | 3.5 |
| tartaric acid (2.5 grams) | |
| Pure citric acid in granular form (four | 3.8 |
| grams) | |
| Pure tartaric acid in granular form (4 | 3.4 |
| grams) | |
| Mixture of citric acid (2 grams) and tartaric | 3.6 |
| acid (2 grams) | |

Also, through experimentation, it was determined that potassium hydroxide can be effective in raising pH as shown in the following Table 2:

TABLE 2

| Amount of Potassium Hydroxide Added | |
|---|---|
| to 600 Milliliters of Urine | Resulting pH |
| 3 grams | 12.3 |
| 4 grams | 12.6 |
| 5 grams | 12.7 |

Pure potassium hydroxide cannot be shipped through the mail or through parcel or air carriers because it is too dangerous. However, it is possible to create a 1/10 mixture of potassium hydroxide with an inert chemical that can be shipped via the mail or through parcel or air carriers.

Referring to the preceding tables, it should be noted that the present invention is capable of preventing odors in a urine solution even when less than 1% of chemicals are added to pure urine. The present invention is also capable of preventing odors when less than 2%, less than 3%, less than 4%, less than 5%, less than 8%, less than 10%, less than 15%, less than 20%, less than 25%, less than 30%, less than 35%, less than 40%, and less than 50% of chemicals are added to a pure solution of urine.

Other experiments have shown that adding a fragrance to the urine solution can provide additional benefits in reducing or masking odors. Examples of fragrances that may be used include, but are not limited to, ______. The fragrance can be in a liquid form is sprayed into the particulate to be added to the urine so that it is added to the urine contemporaneously with the introduction of the pH altering composition. Alternatively, the fragrance can be liquid or solid that is added to the urine solution separately, but contemporaneously as the particulate. This fragrance can be in other forms and added in other ways to the urine solution in ways capable of being understood by someone skilled in the art. Of course, the particulate and the fragrance are most effective if they are water soluable.

Broadly, then, one aspect of this invention is a method of for reducing decomposition odors in a volume of liquid urine solution, comprising mixing the liquid urine solution with a selected quantity of a compound to form a treated volume such that any significant growth of bacteria selected from a group consisting of Escherichia coli, Proteus vulgaris and Pseudomonas in the treated volume is inhibited.

This method may use an acidic compound and wherein the selected quantity is such that the treated volume has a pH of less than about 5.5, but more specifically such that a volume of urine when mixed with the compound will have a pH of less than about 4.0. It is desireable that the compound is a non-toxic, food grade acid, such as one selected from a group consisting of citric acid, succinic acid, tartaric acid and mixtures thereof. The food grade acid may be in a dry particulate for mixture into the urine.

Alternatively, the method may employ an alkaline compound and wherein the selected quantity is such that the treated volume has a pH of more than about 11.5. This alkaline compound may, for example, be potassium hydroxide. Also, by adding a bacteria-killing chemical, such as chlorine, that sufficiently kills, i.e. inhibits, these bacteria, in may be possible to reduce odors without changing the pH.

In addition, a fragrance may be introduced into the liquid urine solution as part of the method. While the fragrance may be introduced at any time, one aspect of the exemplary embodiments is to introduce the fragrance contemporaneously with the introduction of the compound into the urine. Sample fragrances may be as desired, but particular fragrances tested to have good results were sea breeze, lemon grass, lavender and palamarosa.m Each of the fragrances was a concentrated oil-base liquid that was diluted in mineral oil. The diluted oil was applied by an atomizer to a granular mix of citric acid and tartaric acid. Naturally, the dilution ratio and amount of fragrance applied may vary as desired as should be recognized by the ordinarily skilled artisan.

Furthermore, a method of controlling decomposition odors from a collected volume of urine from a human patient is also contemplated by this invention. This method may include, in any order, the steps of: (1) connecting the patient to a urine collection system having a collection container whereby production of urine from the patient will be collected and stored in the collection container; and (2) placing in the collection container a selected quantity of a compound to form a treated volume such that any significant growth of bacteria selected from a group consisting of Escherichia coli, Proteus vulgaris and Pseudomonas in the treated volume is inhibited. This method then includes the step of allowing the production of urine from the patient to be collected and stored in the collection container whereby, as said urine is collected and stored, said compound mixes with said urine. In this method, the compound may be selected to be an acidic compound as described above wherein the selected quantity is such that a volume of urine of no more than 600 milliliters when mixed with the compound will have a pH of less than about 5.5, or more specifically a pH of less that about 4.0. Alternatively, the compound may be an alkaline compound as described above wherein the selected quantity is such that a volume of urine of no more than 600 milliliters when mixed with the compound will have a pH of more than about 11.5. In this method, a fragrance may also be introduced into the liquid urine solution.

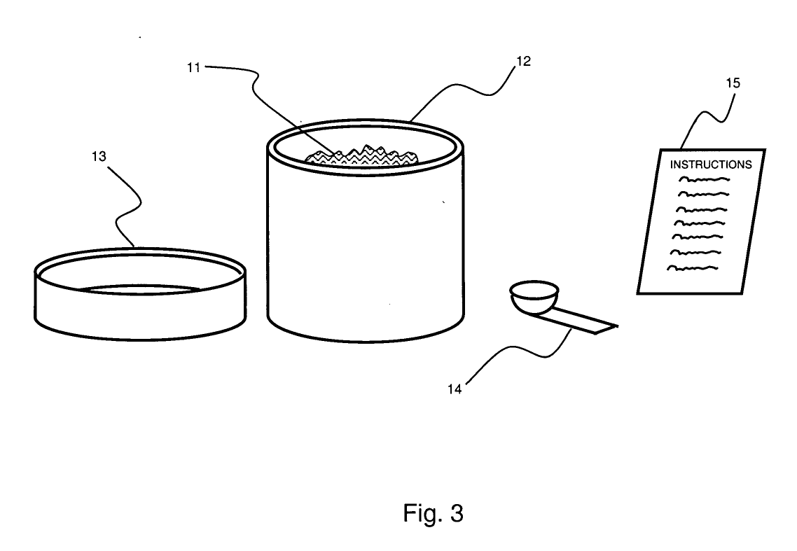

Referring now to FIG. 3, it should be appreciated that the method of preventing the decomposition of urine, as described above, may be implemented using a kit adapted for use in the home or in a medical care facility. The kit includes a pH-changing chemical shown at 11, a chemical storage container shown at 12, a lid for the storage container shown at 13, a measuring spoon shown at 14, and a set of instructions shown at 15. In one embodiment, the chemical composition 11, is chosen from the preceding tables. However, this chemical can be of any other composition or form that has similar microbe exterminating and/or pH modifying capabilities capable of being known by anyone skilled in the art.

Referring still to FIG. 3, in one embodiment the container 12 is a plastic jar made of injection molded polyethylene capable of accepting a screw-on polyethylene lid 13. The kit includes a measuring spoon (or scoop) 14 that has been sized, based on the formulation and type of chemical composition, to allow most users to simply put an integral number, such as one or two level spoonful(s) of the chemical composition 11, into the urine collection device, which might be a bottle, a bag, or any other device capable of holding nighttime's production of human urine. By using such a simple system, the instructions, 15 may be as straightforward as:
1. Place 1 level spoonful of granules in the collection bottle using the spoon provided.
2. Try the system for one night.
3. If the odor is not satisfactorily eliminated, increase the dosage to 1 and one half level spoonfuls. You might need this higher dosage if you produce more than 600 ml of urine per night or if your urine has a pH of greater than 7.5.
4. After the overnight collection is discarded, thoroughly rinse the collection bottle and set it in an inverted position so that the inside dries before the next use.

Accordingly and in addition, the invention contemplates acidic compositions such as described above, that are adapted to be placed in a volume of collected urine. These compositions generally are in the form of an admixture of a non-toxic, food grade acid compound and a fragrance wherein said admixture is in a dry particulate form and is substantially soluable in an aqueous environment. The non-toxic, food grade acid is selected from a group consisting of citric acid, succinic acid, tartaric acid and mixtures thereof.

While the principles of the disclosure have been described above in connection with specific apparatuses and methods, it is to be clearly understood that this description is made only by way of example and not as limitation on the scope of the invention.

I claim:

1. A method for reducing decomposition odors in a volume of liquid urine solution, comprising mixing the liquid urine solution with a selected quantity of a compound to form a treated volume such that any significant growth of bacteria selected from a group consisting of Escherichia coli, Proteus vulgaris and Pseudomonas in the treated volume is inhibited.

2. A method according to claim 1 wherein said compound is an acidic compound and wherein the selected quantity is such that the treated volume has a pH of less than about 5.5.

3. A method according to claim 2 wherein the selected quantity is such that a volume of urine when mixed with the compound will have a pH of less than about 4.0.

4. A method according to claim 2 wherein said compound is a non-toxic, food grade acid.

5. A method according to claim 4 wherein said non-toxic, food grade acid is selected from a group consisting of citric acid, succinic acid, tartaric acid and mixtures thereof.

6. A method according to claim 4 wherein said non-toxic, food grade acid is mixed with the liquid urine as a dry particulate.

7. A method according to claim 1 wherein said compound is an alkaline compound and wherein the selected quantity is such that the treated volume has a pH of more than about 11.5.

8. A method according to claim 7 wherein said alkaline compound is potassium hydroxide.

9. A method according to claim 1 including introducing a fragrance into the liquid urine solution.

10. A method according to claim 9 wherein the introduction of the fragrance occurs contemporaneously with the introduction of said compound.

11. A method of controlling decomposition odors from a collected volume of urine from a patient, comprising:
(A) in any order:
(1) connecting the patient to a urine collection system having a collection container whereby production of urine from the patient will be collected and stored in the collection container;
(2) placing in the collection container a selected quantity of a compound to form a treated volume such that any significant growth of bacteria selected from a group consisting of Escherichia coli, Proteus vulgaris and Pseudomonas in the treated volume is inhibited; and
(B) allowing the production of urine from the patient to be collected and stored in the collection container whereby, as said urine is collected and stored, said compound mixes with said urine.

12. A method according to claim 11 wherein said compound is an acidic compound and wherein the selected quantity is such that a volume of urine of no more than 600 milliliters when mixed with the compound will have a pH of less than about 5.5.

13. A method according to claim 11 wherein said compound is an acidic compound and wherein the selected quantity is such that a volume of urine of no more than 600 milliliters when mixed with the compound will have a pH of less than about 4.0.

14. A method according to claim 12 wherein said compound is a non-toxic, food grade acid.

15. A method according to claim 14 wherein said non-toxic, food grade acid is selected from a group consisting of citric acid, succinic acid, tartaric acid and mixtures thereof.

16. A method according to claim 14 wherein said non-toxic, food grade acid is mixed with the liquid urine as a dry particulate.

17. A method according to claim 11 wherein said compound is an alkaline compound and wherein the selected quantity is such that a volume of urine of no more than 600 milliliters when mixed with the compound will have a pH of more than about 11.5.

18. A method according to claim 17 wherein said alkaline compound is potassium hydroxide.

19. A method according to claim 11 including during step (A) the additional step of introducing a fragrance into the liquid urine solution.

21. A method for reducing decomposition odors in a volume of liquid urine solution, comprising altering the pH of the urine solution by introducing into the liquid urine solution a selected quantity of either an acidic compound or an alkaline compound to form a treated volume such that the pH of the treated volume retards any significant growth of bacteria selected from a group consisting of Escherichia coli, Proteus vulgaris and Pseudomonas.

22. A method according to claim 21 wherein the treated volume has a pH of less than about 5.5.

23. A method according to claim 22 wherein the treated volume has a pH of less than about 4.0.

24. A method according to claim 21 wherein the treated volume has a pH of more than about 11.5.

25. A method according to claim 24 wherein the treated volume has a pH of more than about 12.0.

26. A composition adapted to be placed in a volume of collected urine, comprising an admixture of a non-toxic, food grade acid compound and a fragrance wherein said admixture is in a dry particulate form and is substantially soluable in an aqueous environment.

27. A composition according to claim 26 wherein said non-toxic, food grade acid is selected from a group consisting of citric acid, succinic acid, tartaric acid and mixtures thereof.

28. A kit for use in reducing decomposition odors in a volume of liquid urine solution, comprising;
(A) a container including a lid and having a quantity of a compound therein that is either an acidic compound or an alkaline compound;
(B) a scoop having a selected volume “V” such that the volume nV of said compound, where n is 1 or 2, when mixed with about 600 milliliters of human urine, will form a treated volume of urine that has a pH of less than about 5.5 or more than about 11.5.

29. A kit according to claim 28 wherein the selected volume “V” is such that the volume nV of said compound, where n is 1 or 2, when mixed with about 600 milliliters of human urine, will form a treated volume of urine that has a pH of less than about 4.0 or more than about 12.0.

30. A kit according to claim 28 wherein said compound is an acidic compound.

31. A kit according to claim 30 wherein said compound is a non-toxic, food grade acid.

32. A kit according to claim 31 wherein said non-toxic, food grade acid is selected from a group consisting of citric acid, succinic acid, tartaric acid and mixtures thereof.

33. A kit according to claim 28 wherein said compound is an alkaline compound.

34. A kit according to claim 33 wherein said alkaline compound is potassium hydroxide.

35. A kit according to claim 28 wherein said compound is in the form of a dry particulate.

36. A kit according to claim 28 wherein said compound includes a fragrance.
